(19) United States
(12) Patent Application Publication
(10) Pub. No.: US 2007/0219267 A1
(43) Pub. Date: Sep. 20, 2007

(54) Product for treating warts

(76) Inventor: George R. Royen, Toledo, OH (US)

Correspondence Address: George R. Royer, Suite 416, 316 N. Michigan Street, Toledo, OH, 43624, US

(21) Appl. No.: 11/384,125

(22) Filed: Mar. 20, 2006

(51) Int. Cl. A61K 31/355; A61K 31/19

(52) U.S. Cl. 514/458; 514/557

(57) ABSTRACT

The subject invention is product for amelioration, removal, or other treatment of warts comprising a combination of vinegar and vitamin E, which product may be emulsified in a cream form or a liquid form, or other appropriate form, wherein it is contemplated that said resultant substance be applied directly to the wart's outer surface in one or more applications.

### DISCUSSION OF PRIOR ART AND BACKGROUND OF THE INVENTION

Warts are generally generated from a virus, with the exact type of virus varying from one type of wart to another. Various means are presently used to treat warts including various medicinal ointments, laser treatment, and other medical or surgical remedies. The problems with such known remedies are that invariably most warts reappear some period of time after such treatment. Therefore, there is a need for a more permanent remedy in light of this situation in the art.

### OBJECTS OF THE INVENTION

It is an object of this invention to provide an improved method of removing or treating warts from the skin;

It is also an object of this invention to provide an removal of warts;

Another object of this invention is to provide an improved method of removing warts safely;

Yet another object of this invention is to provide an improved method of removing, or treating warts;

It is also an object of the subject invention to provide a more safe product for wart removal or treatment of warts;

Other and further objects of this invention will become apparent from reading of the claims taken in conjunction with the claims.

### DESCRIPTION OF GENERAL EMBODIMENT AND SUMMARY OF THE INVENTION

The subject invention is product for amelioration, removal, or other treatment of warts comprising a combination of vinegar and vitamin E, which product may be emulsified in a cream form or a liquid form, or other appropriate form, wherein it is contemplated that said resultant substance be applied directly to the wart's outer surface in one or more applications.

Alternately stated, the subject product is a mixture of vitamin E and vinegar intermixed in emulsified form or any other form such as a liquid, paste, ointment, or other forms, and then applied in any manner to the outer surface of the wart, such applications varying any where from one or more.

It may be desirable to maintain the resultant described product immediately over the wart to use some form of covering thereover, such as a protective bandage on the like. Alternately the resultant product may be rubbed over the wart surface in order to improve the absorption of the product on the surface of the wart.

### DESCRIPTION OF SPECIFIC EMBODIMENT

The following description of the subject invention covers one such embodiment of the subject invention and description of a singular embodiment shall not be considered as limiting the scope of the subject invention to as set forth to the claims herein.

The subject invention is product for amelioration, removal, or other treatment of warts comprising a combination of vinegar and vitamin E, which product may be emulsified in a cream form or a liquid form, or other appropriate form, wherein it is contemplated that said resultant substance be applied directly to the wart's outer surface in one or more applications.

Alternately stated, the subject product is a mixture of vitamin E and vinegar intermixed in emulsified form or any other form such as a liquid, paste, ointment, or other forms, and then applied in any manner to the outer surface of the wart, such applications varying any where from one or more.

It may be desirable to maintain the resultant described product immediately over the wart to use some form of covering thereover, such as a protective bandage on the like. Alternately the resultant product may be rubbed over the wart surface in order to improve the absorption of the product on the surface of the wart.

The subject invention involves is an improved method for helping remove, treat, or ameliorate a wart on the surface of the skin, generally including the steps for intermixing vitamin E and vinegar and applying the final product at various intervals to the outer surface of a wart.

The general method of preparing the product generally comprises the following general steps:

1) taking a quantity of vitamin E and intermixing same with a quantity of vinegar;

2) applying such mixture to the outer surface of said wart.

In alternate forms of the subject invention, vitamin E may be used in a cream base, as opposed to oil or liquid and which cream form is then intermixed with vinegar for a more paste-like or ointment-like form for a more effective application and result. The cream may be itself a vitamin E cream, which is an emulsified form with the vinegar intermixed into the cream.

In applying the resultant mixture it may be advisable to use a bandage or some external cover to keep the mixture over the wart for a period of time or apply the product by rubbing the external surface of the wart with the resultant product for better absorption.

The product works more effectively if the product described remains for a significant time on the outer surface of the wart or warts.

In summary, the subject invention can be described as a product for the treatment of a wart comprising a mixture of vinegar and vitamin E.

In further summary, the subject invention can be described as a process for treatment and removal of a wart on the skin of an individual comprising of the steps of:
(a) mixing vitamin E and vinegar into a resultant product;
(b) applying said resultant product or vitamin E and vinegar over the surface of the wart.

In still further summary, the subject invention can be described as a process for treatment and removal of a wart comprising the steps of:
(a) mixing vitamin E and vinegar into a resultant product;
(b) adding said product of vitamin E and vinegar to a cream base and intermixing
   vitamin E and vinegar in such cream.

1. A product for the treatment of a wart comprising a mixture of vinegar and vitamin E.

2. A process for treatment and removal of a wart on the skin of an individual comprising the steps of:
(a) mixing vitamin E and vinegar into a resultant product;
(b) applying said resultant product or vitamin E and vinegar over the surface of the wart.

3. A process for treatment and removal of a wart comprising the steps of:
(a) mixing vitamin E and vinegar into a resultant product;
(b) adding said product of vitamin E and vinegar to a cream base and intermixing vitamin E and vinegar in such cream.
